(19) United States
(12) Patent Application Publication
(10) Pub. No.: US 2005/0158377 A1
(43) Pub. Date: Jul. 21, 2005

(54) Dermatologic soft gel compositions

(76) Inventor: Karl F. Popp, New York, NY (US)

Correspondence Address: NATH & ASSOCIATES, PLLC, Sixth Floor, 1030 15th Street, N.W., Washington, DC, 20005, US

(21) Appl. No.: 11/037,120

(22) Filed: Jan. 19, 2005

Related U.S. Application Data
(60) Provisional application No. 60/537,288, filed on Jan. 20, 2004.

(51) Int. Cl. A61K 9/48

(52) U.S. Cl. 424/451

(57) ABSTRACT

Orally administrable softgels or soft gelatin capsules and fill compositions therefore for use in treating various dermatological conditions. These compositions are also particularly useful for treating children or patients of at least 55 years of age.

This application claims priority to U.S. Provisional Patent Application Ser. No. 60/537,288, filed on Jan. 20, 2004, the contents of which are hereby incorporated by reference in their entirety.

### FIELD OF THE INVENTION

The present subject matter relates to orally administrable softgels or soft gelatin capsules and fill compositions therefore for use in treating various dermatological conditions. These compositions are particularly useful for treating children, patients of at least 55 years of age, and females.

### BACKGROUND OF THE INVENTION

The topical administration of various pharmacologically active agents to treat various dermatological disorders has long been known in the art. The accessibility of the skin and the opportunity it provides for application of topical preparations over a prolonged period of time have resulted in an increasing use of topical drug delivery systems over the past number of years. Typically, these topical dosage forms can be in liquid, semisolid, or solid form.

Drugs have typically been applied to the skin in this manner to elicit one or more of four general effects: an effect on the skin surface, an effect within the stratum corneum, a more deep-seated effect requiring penetration into the epidermis and dermis, or a systemic effect resulting from delivery of a sufficient amount of drug through the epidermis and the dermis to the vasculature to produce therapeutic systemic concentrations.

However, the penetration of a drug into the viable epidermis and dermis when applied in a topical dosage form may sometimes be difficult to achieve. Further, even if drug penetration is achieved, the drug may only be delivered to the local area where the composition is applied, rather than regionally or systemically. Accordingly, topical compositions are generally not optimal in treating many dermatological disorders that exhibit certain regional or systemic effects.

Topical pharmaceutical dosage forms may have the further disadvantage of exhibiting side effects on application, such as irritation to sensitive skin areas. Such irritation is often due to the presence of preservatives to maintain the stability of the active agent in the topical dosage form. Maintaining drug stability in topical compositions at times can be a very difficult endeavor, making preservatives a very common and necessary ingredient in many topical compositions.

Further, topical compositions at times have to remain in contact with the skin for an extended period of time to release sufficient amounts of the active agent to the skin and exert the desired pharmacological effect against a dermatological disorder. However, it may be difficult to formulate a topical composition that remains on the skin for this extended period of time without wearing or rubbing off during the wearers regular daily activities. Further, topical compositions that are sufficiently robust to remain on the skin for extended periods of time often have disadvantages in that they may not be readily absorbed by the skin, they may tend to block skin pores, they may be greasy in nature, and they may be difficult to wash off the skin.

To overcome some of these problems associated with certain topical treatments of dermatological disorders, many drugs may be administered in an oral dosage form. The most common oral dosage forms are tablets and capsules. Tablets and capsules may be prepared from the compression of solid ingredients, in powder form or otherwise. However, an oral dosage form such as a tablet or capsule formed via compression oftentimes results in a large amount of degradates of the active ingredient.

Further, solid oral dosage forms may cause irritation upon administration due to the presence of the active agent in a powdery, crystal form. This powdery, crystal form of the active ingredient likewise may make it difficult to achieve an optimal, controlled dissolution and absorption of the active agent after administration. It is oftentimes difficult to attain a consistent bioavailability of the active agent due to this powdery crystal form.

Most tablets also require the use of a diluent, or a bulking agent, to make the tablet a practical size for compression. Similarly, tablets oftentimes contain other excipients such as binders, lubricants, glidants, and disintegrants to permit formation of the tablet, as well as to aid in drug delivery. However, the presence of these additional ingredients may have an adverse effect on both the patient and the stability of the active ingredients, depending on the agent used.

Additionally, certain hard tablets and capsules are poor delivery devices for hydrophobic drugs. Hydrophobic drugs generally do not dissolve readily in water, gastric fluid, or intestinal fluid. When they are compounded in solid dosage forms, the dissolution rate may be slow, absorption may vary, and the bioavailability may be incomplete.

Hard tablets and hard capsules are also difficult for certain patients, particularly certain young and old patients, as well as female patients, to swallow. This is due to their hard, compact nature, which results in a rough exterior that may easily get caught in the mouth or throat. Accordingly, there remains a need for an additional dosage form easily administrable to young, old, and female human patients that is effective for the treatment of dermatological disorders.

Soft gel capsules, or softgels, are known in the art as alternative dosage forms to those described above, but not necessarily for the treatment of dermatological disorders. For example, U.S. Pat. No. 5,587,149 discloses such a softgel formulation for water-soluble active ingredients, such as ascorbic acid (vitamin C), where the fill material comprises an emulsion of which a first phase includes polyethylene glycol (into which the water-soluble active ingredient is dissolved) and the second phase includes a silicone fluid.

Likewise, U.S. Pat. No. 6,251,426 discloses a soft gelatin capsule that contains a highly concentrated solution of ibuprofen. However, this patent does not disclose the ability of softgels to deliver active agents useful in treating dermatological disorders.

U.S. Pat. No. 5,200,191 discloses a softgel composition containing retinol for topical application to the skin. The disclosed softgel provides a single use method for dispensing the product, wherein the softgel contains a twist-off or other removable feature at one end for dispensing the fill material encompassed therein. However, since the active agent in the disclosed softgel is applied topically to the skin, this dosage form is very similar to the topical dosage forms previously discussed.

One oral softgel known in the art for the treatment of dermatological disorders is Accutane®, a softgel available from Hoffmann-La Roche, New Jersey, containing the active ingredient isotretinoin, a known retinoid. The soft gel dosage form is used to protect the isotretinoin during manufacturing, as retinoids as a class of compounds must be protected from oxygen to prevent oxidation. However, this softgel composition does not possess any advantages over, e.g., a topical composition containing isotretinoin with respect to the actual delivery of the drug to a patient. In fact, since the isotretinoin is contained in the Accutane® softgel in a liquid suspension, it has a half life after administration of about 90 hours, resulting in a high possibility of adverse side effects.

Accordingly, there remains a need in the art for methods of treating certain dermatological disorders by administering a composition that can effectively deliver an active agent to the body for the treatment of the dermatological disorder. Such a method would provide an alternative to topical dosage forms and compressed oral dosage forms such as tablets and capsules by effectively administering a drug orally for the treatment of dermatological disorders. There further remains a need for treating dermatological disorders in young, old, and female patients by administering an oral composition that is easily and readily taken by these patient groups. The present subject matter addresses these needs.

### SUMMARY OF THE INVENTION

The present subject matter relates generally to a method of treating a dermatological disorder in a mammal. This method is achieved by administering to the mammal a soft gel capsule providing a therapeutically effective amount of a pharmacologically active agent. The soft gel capsule preferably comprises an internal, non-aqueous liquid phase and an external gelatin and/or soft cellulose layer. The internal, non-aqueous liquid phase may comprise a solution or suspension of the pharmacologically active agent having a purity of at least 90% and a concentration of degradation product(s) less than about 10% of the starting concentration of the pharmacologically active agent. This purity and concentration of degradation product(s) of the active agent are preferably sufficient to permit safe treatment of the dermatological disorder and provide improved bioavailability of the pharmacologically active agent.

In a preferred embodiment, the pharmacologically active agent is selected from the group consisting of antibiotics, antiinfectives, antimycotic agents, steroids, antihistamines, antiparasitic agents, immunomodulators, antisense agents, antiviral agents, treatments for hypo- and hyper-skin pigmentation disorders, antipsoriatic agents, keratolytic agents, immunosuppressants, DNA synthesis inhibitors, cytotoxic agents, antithyroid agents, monoclonal antibody regulators, TNF alpha antagonists, immunoglobulins, metabolic regulators, antiangiogenic agents, kinase regulators, hormones, photodynamic agents, protease inhibitors, anxiolytics, cell growth regulators, enzymes, prostaglandins, peptides, analgesics, salts thereof, derivatives thereof, and mixtures thereof.

In another preferred embodiment, the present subject matter relates to a method of treating a dermatological disorder in a mammal, comprising:
orally administering to said mammal a soft gel capsule providing improved bioavailability of a pharmacologically active agent comprising:
an internal, non-aqueous liquid phase comprising a solution or suspension of a single, hydrophobic, pharmacologically active agent effective to treat said dermatological disorder having a purity of at least 90% and a concentration of degradation product(s) less than about 10% of the starting concentration of said hydrophobic pharmacologically active agent, wherein said purity and concentration of degradation product(s) are sufficient to permit safe treatment of said dermatological disorder; and
an external gelatin layer comprising gelatin, soft cellulose, or a mixture thereof and additional components selected from the group consisting of an additional gelling agent, a plasticizer, water, a colorant, an antioxidant, a flavorant, and mixtures thereof;
wherein said hydrophobic pharmacologically active agent is selected from the group consisting of antiinfectives, steroids, a salt thereof, a derivative thereof, and mixtures thereof.

In yet another preferred embodiment, the present subject matter relates to a method of treating a dermatological disorder in a mammal, comprising:
orally administering to said mammal a soft gel capsule providing improved bioavailability of a pharmacologically active agent comprising:
an internal, non-aqueous liquid phase having a pH of from about 3 to about 9 when combined with an aqueous medium comprising a solution or suspension of a single, hydrophobic, pharmacologically active agent effective to treat said dermatological disorder and one or more fatty acids or derivatives thereof selected from the group consisting of omega-3 fatty acids, DHA, docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, said single pharmacologically active agent comprising a hydrophobic antiinfective agent or a salt or derivative thereof having a purity of at least 90% and a concentration of degradation product(s) less than about 10% of the starting concentration of said hydrophobic antibiotic agent, wherein said purity and concentration of degradation product(s) are sufficient to permit safe treatment of said dermatological disorder; and
an external gelatin layer comprising gelatin and additional components selected from the group consisting of an additional gelling agent, a plasticizer, water, a colorant, an antioxidant, a flavorant, and mixtures thereof.

In still another preferred embodiment, the present subject matter relates to a method of treating a dermatological disorder in a mammal, comprising:
orally administering to said mammal a soft gel capsule providing improved bioavailability of doxycycline or a salt or derivative thereof comprising:
an internal, non-aqueous liquid phase having a pH of from about 3 to about 9 when combined with an aqueous medium comprising a solution or suspension of doxycycline or a salt or derivative thereof as a sole active ingredient effective to treat said dermatological disorder and one or more fatty acids or derivatives thereof selected from the group consisting of omega-3 fatty acids, DHA, docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, said doxycycline having a purity of at least 95% and a concentration of degradation product(s) less than about 5% of the starting concentration of said doxycycline, wherein said purity and concentration of degradation product(s) are sufficient to permit safe treatment of said dermatological disorder; and
an external gelatin layer comprising gelatin and additional components selected from the group consisting of an additional gelling agent, a plasticizer, water, a colorant, an antioxidant, a flavorant, and mixtures thereof.

In an alternative preferred embodiment, the present subject matter relates to a method of treating a dermatological disorder in a mammal, comprising:
orally administering to said mammal a soft gel capsule providing improved bioavailability of a hydrophobic pharmacologically active agent comprising:
an internal, non-aqueous liquid phase comprising a solution or suspension of a single hydrophobic pharmacologically active agent or a salt or derivative thereof effective to treat said dermatological disorder and one or more fatty acids or derivatives thereof selected from the group consisting of omega-3 fatty acids, docosahexaenoic acid (DHA), docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, said hydrophobic pharmacologically active agent having a purity of at least 90% and a concentration of degradation product(s) less than about 10% of the starting concentration of said hydrophobic pharmacologically active agent, wherein said purity and concentration of degradation product(s) are sufficient to permit safe treatment of said dermatological disorder; and
an external gelatin layer comprising gelatin and additional components selected from the group consisting of an additional gelling agent, a plasticizer, water, a colorant, an antioxidant, a flavorant, and mixtures thereof.

In a further alternative embodiment, the present subject matter relates to a method for treating a human patient having an age in excess of at least 55 years, comprising:
orally administering to said human patient in need thereof a soft gel capsule providing improved bioavailability of a pharmacologically active agent comprising:
an internal, non-aqueous liquid phase comprising a solution or suspension of a pharmacologically active agent having a purity of at least 90% and a concentration of degradation product(s) less than about 10% of the starting concentration of said pharmacologically active agent and one or more fatty acids or derivatives thereof, wherein said purity and concentration of degradation product(s) are sufficient to permit safe treatment of said human patient and provide improved bioavailability of said pharmacologically active agent; and
an external gelatin and/or soft cellulose layer;
wherein said pharmacologically active agent is selected from the group consisting of antibiotics, antiinfectives, antimycotic agents, steroids, antihistamines, antiparasitic agents, immunomodulators, antisense agents, antiviral agents, treatments for hypo- and hyper-skin pigmentation disorders, antipsoriatic agents, keratolytic agents, immunosuppressants, DNA synthesis inhibitors, cytotoxic agents, antithyroid agents, monoclonal antibody regulators, TNF alpha antagonists, immunoglobulins, metabolic regulators, antiangiogenic agents, kinase regulators, hormones, photodynamic agents, protease inhibitors, anxiolytics, cell growth regulators, enzymes, prostaglandins, peptides, analgesics, salts thereof, derivatives thereof, and mixtures thereof.

In yet another alternative embodiment, the present subject matter relates to a method of treating a dermatological disorder in a human patient having an age in excess of at least 55 years, comprising:
orally administering to said human patient in need thereof a soft gel capsule providing improved bioavailability of a tetracycline comprising:
an internal, non-aqueous liquid phase having a pH of from about 3 to about 9 when combined with an aqueous medium comprising a solution or suspension of a tetracycline or a salt or derivative thereof as a sole active ingredient effective to treat said dermatological disorder and one or more fatty acids or derivatives thereof selected from the group consisting of omega-3 fatty acids, docosahexaenoic acid (DHA), docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, said tetracycline having a purity of at least 90% and a concentration of degradation product(s) less than about 10% of the starting concentration of said tetracycline, wherein said purity and concentration of degradation product(s) are sufficient to permit safe treatment of said human patient; and
an external gelatin layer comprising gelatin and additional components selected from the group consisting of an additional gelling agent, a plasticizer, water, a colorant, an antioxidant, a flavorant, and mixtures thereof.

In still another preferred embodiment, the present subject matter relates to a soft gel capsule suitable for oral administration to a human and providing improved bioavailability of a dermatologically effective active agent comprising:
an internal, non-aqueous liquid phase having a pH of from about 3 to about 9 when combined with an aqueous medium comprising a solution or suspension of a dermatologically effective active agent or a salt or derivative thereof and one or more fatty acids or derivatives thereof selected from the group consisting of omega-3 fatty acids, DHA, docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, said dermatologically active agent having a purity of at least 90% and a concentration of degradation product(s) less than about 10% of the starting concentration of said dermatologically active agent, wherein said purity and concentration of degradation product(s) are sufficient to permit safe treatment of said human patient; and
an external gelatin layer comprising gelatin and additional components selected from the group consisting of an additional gelling agent, a plasticizer, water, a colorant, an antioxidant, a flavorant, and mixtures thereof.

In a further preferred embodiment, the present subject matter relates to a soft gel capsule suitable for oral administration to a human and providing improved bioavailability of a tetracycline comprising:
an internal, non-aqueous liquid phase having a pH of from about 3 to about 9 when combined with an aqueous medium comprising a solution or suspension of a tetracycline or a salt or derivative thereof as a sole active ingredient effective to treat a dermatological disorder and one or more fatty acids or derivatives thereof selected from the group consisting of omega-3 fatty acids, docosahexaenoic acid (DHA), docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, said tetracycline having a purity of at least 90% and a concentration of degradation product(s) less than about 10% of the starting concentration of said tetracycline, wherein said purity and concentration of degradation product(s) are sufficient to permit safe treatment of said human patient; and
an external gelatin layer comprising gelatin and additional components selected from the group consisting of an additional gelling agent, a plasticizer, water, a colorant, an antioxidant, a flavorant, and mixtures thereof.

### DETAILED DESCRIPTION OF THE INVENTION

Definitions

As used herein, “administering” refers to providing a composition orally or to a body orifice of a patient being treated. The term administering as used herein excludes providing a composition to a patient either intravenously or via inhalation.

As used herein, a “controlled release” refers to a release rate that is different from the pharmacologically active agent's normal release rate. Accordingly, this term indicates that the release rate of the pharmacologically active agent has been modified to achieve a delayed, sustained, or extended release in comparison to the agent's normal release rate.

As used herein, “degradation products” refers to the product(s) produced by decomposition of one or more of the active ingredients of the present compositions.

The phrase “effective amount”, as used herein, means an amount of a composition or component thereof sufficient enough to positively modify the disorder to be treated but low enough to avoid secondary infections that cause a need for additional treatments beyond those contemplated herein. Effective amounts will vary with the particular disorder or disorders being treated, the severity of the disorder, the duration of the treatment, the specific components of the composition being used, the weight, tolerance, and other physical attributes of the patient being treated, and like factors as are known by health-care providers, including physicians.

As used herein, a “hard” oral dosage form refers to a solid oral drug delivery system formed for example via compression, direct or otherwise, granulation, and/or spray drying. For example, such a hard oral dosage form can be formed by compression of one or more powdery substances. Hard tablets, caplets, and pellets included in capsules are non-limiting examples of such hard oral dosage forms.

As used herein, “pharmaceutically acceptable salts” refers to salts of the active compound(s) which possess the same pharmacological activity as the active compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, naturally and synthetically derived amino acids.

If organic bases are used, poorly volatile bases are preferably employed, for example low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine. Further poorly volatile bases which may be mentioned are, for example, ethylenediamine, glucosamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine.

Salts of quaternary ammonium hydroxides such as trimethylbenzylammonium hydroxide, tetramethylammonium hydroxide, or tetraethylammonium hydroxide can also by used, as can guanidine and its derivatives, in particular its alkylation products. However, it is also possible to employ as salt-forming agents, for example, low molecular weight alkylamines such as methylamine, ethylamine, or triethylamine. Suitable salts for the compounds to be employed according to the present subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained.

As used herein, “softgel”, “soft gel”, and “soft gelatin” can be used interchangeably and all refer to capsules having a one-piece, hermetically sealed shell wall, or external layer, filled with oils and/or other aqueous or non-aqueous liquids, plus solids dispersed therein, either in solution or otherwise.

As used herein, “treating” or “treatment” means the prevention or reduction of severity of symptoms or effect of a dermatological disorder, disease, infection, allergy, reaction, or other dermatological condition.

Other terms as used herein are meant to be defined by their well-known meanings in the art.

Soft Gel Capsules

According to the preferred methods and compositions herein, a softgel, or soft gelatin, capsule is administered to a mammal to provide a therapeutically effective amount of a pharmacologically active agent in order to treat a dermatological disease in said mammal. Preferred methods in this regard relate to methods of treating a dermatological disorder in a mammal comprising administering to said mammal a soft gel capsule providing a therapeutically effective amount of a pharmacologically active agent.

In a preferred embodiment, the soft gel capsule comprises an internal phase and an external phase. The internal phase is preferably an internal, non-aqueous liquid phase comprising a solution or suspension of a pharmacologically active agent or a salt or derivative thereof having a purity of at least 90% and a concentration of degradation product(s) less than about 10% of the starting concentration of the pharmacologically active agent. This purity level and concentration of degradation product(s) are sufficient to permit safe treatment of the dermatological disorder and provide improved bioavailability of the pharmacologically active agent.

In a further preferred embodiment, the external phase is an external gelatin and/or soft cellulose layer.

In another preferred embodiment, the pharmacologically active agent used in the soft gel capsule is selected from the group consisting of antibiotics, antiinfectives, antimycotic agents, steroids, antihistamines, antiparasitic agents, immunomodulators, antisense agents, antiviral agents, treatments for hypo- and hyper-skin pigmentation disorders, antipsoriatic agents, keratolytic agents, immunosuppressants, DNA synthesis inhibitors, cytotoxic agents, antithyroid agents, monoclonal antibody regulators, TNF alpha antagonists, immunoglobulins, metabolic regulators, antiangiogenic agents, kinase regulators, hormones, photodynamic agents, protease inhibitors, anxiolytics, cell growth regulators, enzymes, prostaglandins, peptides, analgesics, salts thereof, derivatives thereof, and mixtures thereof.

In a particularly preferred embodiment, the soft gel capsules are orally administered to the patient.

The soft gel capsules used herein provide distinct advantages over prior art hard oral dosage forms known as useful for orally treating dermatological disorders. For example, despite the need to form the soft gel capsules at a relatively high temperature, the present soft gel capsules oftentimes contain fewer degradates of the active pharmacological agent than a comparable hard tablet or hard capsule due to the use of a lower compression pressure during formation of the soft gel.

Further, since the soft gel capsules are formed using liquid rather than powdery ingredients, the soft gel dosage form unexpectedly provides less irritation and greater active stability than a corresponding hard tablet or hard capsule upon administration to a patient. The use of liquid ingredients in the formation of soft gels may also provide enhanced absorption and dissolution characteristics in comparison with hard tablets or hard capsules, as well as a more consistent bioavailability.

These differences allow the present soft gel capsules to maintain a high purity level and a low concentration of degradation products of the dermatological active ingredient both during formation of the pharmaceutical dosage form and during an extended storage period. Accordingly, the present soft gel capsules may at times contain a higher purity level of dermatological active ingredients than hard tablets and hard capsules, resulting in the improved treatment of various dermatological disorders, especially regionally or systemically.

In preferred embodiments, the present soft gel capsules maintain a concentration of degradation product(s) less than about 7%, more preferably less than about 5%, most preferably less than about 3%, of the starting concentration of the pharmacologically active agent contained therein. In this regard, the present soft gel capsules maintain a concentration of degradation products of the active ingredient contained therein well within the limits for that specific active provided by a regulatory government agency, such as the U.S. Food and Drug Administration (FDA).

Similarly, the present soft gel capsules maintain a purity level of at least 90%, preferably at least 95%, of the pharmacologically active agent contained therein. These advantageous properties permit the enhanced treatment of dermatological disorders using orally administrable pharmaceutical dosage forms.

Further, the ability of the present soft gel capsules to exhibit therapeutic dermatological effects while minimizing the amount of additional excipients at times required by hard tablets and capsules represents a significant improvement over the typical hard oral compositions previously known in the art. As fewer ingredients are present in a composition, the chances of a patient having an adverse reaction to the composition will decrease. The present soft gel capsules, then, are expected to produce an adverse reaction in a low number of patients.

Additionally, the present softgel capsules deliver drugs in solution while offering a solid dosage form. Accordingly, these soft gel capsules are effective delivery systems for hydrophobic drugs, which can be dissolved in a hydrophilic solvent that, when the capsule is crushed, chewed, or dissolved, can release the hydrophobic drug immediately to produce a solution of the drug in gastric juice ready for absorption from the gastrointestinal tract into the blood stream. This can result in the rapid onset of a desired therapeutic effect.

Accordingly, the present soft gel capsules effectively treat dermatological disorders in a patient without exhibiting certain disadvantages at times associated with hard tablets and hard capsules.

The present soft gel capsules likewise correct the disadvantages of using the typical topical dosage forms previously known in the art for the treatment of dermatological disorders. For example, the use of the present soft gels promotes enhanced absorption and dissolution characteristics, a more consistent bioavailability, and a regional or systemic effectiveness of the dermatologically active agents contained therein. In contrast, certain topical dosage forms may at times provide limited penetration of a drug, which may only be delivered to the local area where the composition is applied, rather than regionally or systemically. Further, it is at times difficult to control and/or maximize the absorption, dissolution, and bioavailability characteristics of an active agent administered via many previously known topical dosage forms.

Further, at times it may be difficult to produce a stable topical composition effective for treating various dermatological disorders. Accordingly, these particular topical compositions have a limited shelf life and cannot remain on the market, or in use, for an extended period of time. The present soft gel capsules, in contrast, are very stable compositions, possessing an extended period of storage stability.

In this regard, topical dosage forms often contain additional ingredients such as preservatives to maintain the stability of the active agent in the topical dosage form. However, such additional ingredients at times can often have an adverse impact, such as irritation to sensitive skin, upon administration to a patient. The present soft gel capsules, in contrast, have an enhanced stability of the pharmacologically active agent without needing to use a preservative as an essential component. This unexpectedly provides a reduced incidence of irritation than many of the corresponding topical pharmaceutical dosage forms.

The present soft gel capsules are easily administered orally to a patient suffering from any of a variety of dermatological disorders. In fact, the smooth nature of the external layer of these compositions permits the compositions to be easily swallowed by young patients, i.e. children of between 5 and 20 years old, by patients, sometimes referred to as geriatric patients, between 55 and 90 years old or older, and by female patients, as opposed to male patients. In a preferred embodiment, the present compositions are easily swallowed by children of between 8 and 18 years old. In contrast, previous hard oral dosage forms tend to not be as easily swallowed by these same patients.

Additionally, many of the previous topical compositions at times must remain in contact with the skin for an extended period of time to release sufficient quantities of the active agent to the skin and exert the desired pharmacological effect against a dermatological disorder. This can require either uncomfortable compositions that are sufficiently robust to remain on the skin for extended periods of time or compositions that must be applied multiple times daily, resulting in sub-optimal patient compliance. Accordingly, since the present soft gel capsules are administered orally rather than topically, they may result in an increased patient compliance with the necessary regimen to effectively treat a dermatological disorder.

The present soft gel capsules can be made in a variety of colors, or can have an indicia printed on the exterior after formation, to provide a designation of goods or an indication of the source of the capsule, or the pharmacologically active agent encapsulated therein. The printing material may be any suitable dye or pigment, and the identification may be applied by any known process or machine used for applying some type of identification on softgels, such as those shown, for example, in U.S. Pat. Nos. 2,449,139; 2,623,494; 2,703,047; 2,688,775; 3,124,840; 3,203,347; and 3,333,031, the entire contents of which are hereby incorporated by reference.

External Layer

The present soft gel capsules comprise a smooth external layer as an essential component. The smoothness of this external layer permits the compositions to be easily swallowed by young patients, i.e. children of between 5 and 20 years old, preferably children of between 8 and 18 years old, patients, sometimes referred to as geriatric patients, having an age in excess of at least 55 years, and female patients. In this regard, the external layer may be an external gelatin layer or an external soft cellulose layer. Further, the external layer permits the softgel to be packaged as convenient single-use containers.

The external layer conveys a unique strength and durability to the present soft gel capsules. Additionally, the external layer protects the internal liquid phase, or fill material, from atmospheric oxidation that compromises other oral dosage forms such as hard tablets and hard capsules in terms of potency and shelf life. Further, the external layer may provide a controlled release of the ingredients present in the internal liquid phase.

Preferred external gelatin layers useful herein comprise gelatin and an additional component selected from the group consisting of an additional gelling agent, a plasticizer, water, a colorant, an antioxidant, a flavorant, and mixtures thereof. Similarly, preferred external soft cellulose layers useful herein comprise a cellulose selected from the group consisting of a hydroxypropylmethylcellulose, hydroxypropylcellulose, hydroxyethylcellulose, hydroxymethylcellulose, ethylcellulose, microcrystalline cellulose, and mixtures thereof, and additional components selected from the group consisting of a gelling agent, a salt, a plasticizer, water, a colorant, an antioxidant, a flavorant, and mixtures thereof. A preferred gelling agent in this regard is a carrageenan. Other components known to those of skill in the art as useful in forming gelatin or soft cellulose layers are further contemplated herein.

The external layer of the present soft gel capsules may further comprise any other ingredient listed as suitable for such a dosage form in the “Inactive Ingredient Guide”, U.S. Food and Drug Administration (FDA) Center for Drug Evaluation and Research (CDER) Office of Management, January 1996, the contents of which are hereby incorporated by reference in their entirety.

Since the present smooth external layer is formed from a liquid, it contains a rounded shape with curved edges. Accordingly, the external layer preferably can come in a great variety of rounded sizes and shapes, including but not limited to ovoid, spherical, oblong, tubular, and other special types of shapes. In particularly preferred embodiments, the external layer has an ovoid or spherical shape. The curved edges and rounded shape of the external layer permit easier swallowing of the present soft gel capsules in comparison to hard gelatin capsules. Further, the external layer may be formulated to provide a controlled release and improve palatability of the pharmacologically active agent. For example, the external layer may incorporate phospholipids or polymers or natural gums to entrap the pharmacologically active agent therein to convey desired delayed/controlled release effects. These features contribute to improved patient compliance with and a reduced incidence of side effects upon the administration of a pharmacologically active agent using the present soft gel capsules.

Internal Liquid Phase

The present soft gel capsules additionally comprise an essential internal liquid phase. This internal liquid phase is preferably a non-aqueous liquid phase. When combined with an aqueous medium, the internal liquid phase preferably has a pH of about 3 to about 9.

The internal liquid phase may be a single phase or a mixture of miscible liquids. In preferred embodiments, the internal liquid phase of the present soft gel capsules comprises a solution, suspension, or paste of a pharmacologically active agent and one or more fatty acids or derivatives thereof. Preferred, non-limiting examples of such fatty acids include those selected from the group consisting of fatty acids, esters of fatty acids, ethers of fatty acids, alcohols of fatty acids, and mixtures thereof.

Preferred, non-limiting examples of specific fatty acids useful in the internal liquid phase of the present soft gel capsules include those selected from the group consisting of omega-3 fatty acids, docosahexaenoic acid (DHA), docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof.

The internal liquid phase of the present soft gel capsules may optionally further comprise an additional ingredient selected from the group consisting of peanut oil, hydrogenated peanut oil, castor oil, hydrogenated castor oil, corn oil, olive oil, hydrogenated vegetable oils, silicone oil, soya oil, paraffin oil, cetyl alcohol, cetostearyl alcohol, stearyl alcohol, stearic acid, beeswax, silica dioxide, polyethylene glycol, monoglycerides, diglycerides, triglycerides, poloxamers, silicone oils, and mixtures thereof. Other aqueous or non-aqueous liquids suitable as carriers for pharmacologically active agents are further contemplated herein, with non-aqueous liquids being preferred.

The internal liquid phase of the present soft gel capsules may further comprise any other ingredient listed as suitable for such a dosage form in the “Inactive Ingredient Guide”, U.S. Food and Drug Administration (FDA) Center for Drug Evaluation and Research (CDER) Office of Management, January 1996, the contents of which are hereby incorporated by reference in their entirety.

Pharmacologically Active Agent

An essential component of the internal liquid phase of the present soft gel capsules is a pharmacologically active agent effective for treatment of a dermatological disorder in a patient. The pharmacologically active agent is preferably added to the internal liquid phase in its ionized form, unionized form, or a mixture thereof.

In a preferred embodiment, the pharmacologically active agent is in solution in the internal liquid phase. In an alternative preferred embodiment, the pharmacologically active agent is not in solution in the internal liquid phase. In this regard, the pharmacologically active agent may be in suspension or in a paste in the internal liquid phase.

This pharmacologically active agent is preferably present in the instant compositions at a purity of at least 90% and has a concentration of degradation products less than about 10% of the starting concentration of the pharmacologically active agent. This purity and concentration of degradation products permits safe treatment of a dermatological disorder and provides improved bioavailability of the pharmacologically active agent.

In a preferred embodiment, the present soft gel capsules maintain a concentration of degradation product(s) less than about 7%, more preferably less than about 5%, most preferably less than about 3%, of the starting concentration of the pharmacologically active agent contained therein. In this regard, the present soft gel capsules maintain a concentration of degradation products of the active ingredient contained therein well within the limits for that specific active provided by a regulatory government agency, such as the U.S. FDA.

Similarly, the present soft gel capsules preferably maintain a purity level of at least 95% of the pharmacologically active agent contained therein. These advantageous properties permit the enhanced treatment of dermatological disorders using orally administrable pharmaceutical dosage forms.

Preferred, non-limiting examples of pharmacologically active agents useful in the present methods of treating dermatological disorders include antibiotics, antiinfectives, antimycotic agents, steroids, antihistamines, antiparasitic agents, immunomodulators, antisense agents, antiviral agents, treatments for hypo- and hyper-skin pigmentation disorders, antipsoriatic agents, keratolytic agents, immunosuppressants, DNA synthesis inhibitors, cytotoxic agents, antithyroid agents, monoclonal antibody regulators, TNF alpha antagonists, immunoglobulins, metabolic regulators, antiangiogenic agents, kinase regulators, hormones, photodynamic agents, protease inhibitors, anxiolytics, cell growth regulators, enzymes, prostaglandins, peptides, analgesics, salts thereof, derivatives thereof, and mixtures thereof. In a particularly preferred embodiment, the pharmacologically active agent or salt or derivative thereof is hydrophobic.

In a particularly preferred embodiment, the pharmacologically active agent is an antiinfective agent. Preferred, non-limiting examples of antiinfective agents useful in the present soft gel capsules include those selected from the group consisting of tetracyclines, cephalosporins, β-lactams, polypeptides, sulfa agents, aminoglycosides, macrolides, penicillins, quinolones, amphenicols, lincosamides, ansamycins, nitrofurans, carbapenems, cephamycins, monobactams, ketolides, salts thereof, derivatives thereof, and mixtures thereof.

In an especially preferred embodiment, the antiinfective agent is a tetracycline. Preferred, non-limiting examples of tetracyclines useful herein include those selected from the group consisting of chlortetracycline, clomocycline, demeclocycline, demnocycline, doxycycline, guamecycline, lymecycline, meclocycline, methacycline, minocycline, oxytetracycline, penirnepicycline, pipacycline, rolitetracycline, sanicycline, senocicline, spicycline, tetracycline, salts thereof, derivatives thereof, and a mixture thereof. Doxycycline, a salt thereof, or a derivative thereof is particularly preferred in this regard.

In an alternative preferred embodiment, the antiinfective agent is a cephalosporin. Preferred, non-limiting examples of cephalosporins useful in this regard include those selected from the group consisting of 1-carba (dethia) cephalosporin, cefaclor, cefactor, cefadroxil, cefamandole, cefatrizine, cefazedone, cefazolin, cefepime, cefixime, cefmenoxime, cefmetazole, cefodizime, cefonicid, cefoperazone, ceforanide, cefotaxime, cefotetan, cefotiam, cefoxitin, cefpirnizole, cefpirimide, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile, cephalexin, cephaloglycin, cephaloridine, cephalosporin, cephalothin, cephapirin, cephradine, loracabef, pivcefalexin, salts thereof, derivatives thereof, and mixtures thereof.

In another alternative preferred embodiment, the antiinfective agent is a β-lactam. Preferred, non-limiting examples of β-lactams useful in this regard include those selected from the group consisting of imipenem, meropenem, aztreonam, clavulanic acid, sulbactam, tazobactam, salts thereof, derivatives thereof, and mixtures thereof.

In yet another alternative preferred embodiment, the antiinfective agent is a polypeptide. Preferred, non-limiting examples of polypeptides useful in this regard include those selected from the group consisting of amphomycin, bacitracin, capreomycin, colistin, enduracidin, enviomycin, fusafungine, gramicidin, gramicidin S, mikamycin, polymyxin, polymyxin β-methanesulfonic acid, pristinamycin, ristocetin, teicoplanin, thiostrepton, tuberactinomycin, tyrocidine, tyrothricin, vancomycin, viomycin, virginiamycin, zinc bacitracin, salts thereof, derivatives thereof, and mixtures thereof.

In still another alternative preferred embodiment, the antiinfective agent is a sulfa agent. Preferred, non-limiting examples of sulfa agents useful in this regard include those selected from the group consisting of acetyl sulfamethoxypyrazine, acetyl sulfisoxazole, azosulfamide, benzylsulfamide, chloramine-β, chloramine-T, dichloramine-T, formosulfathiazole, N₂-formyl-sulfisomidine, N₄-β-D-glucosylsulfanilamide, mafenide, 4′-(methyl-sulfamoyl)sulfanilanilide, p-nitrosulfathiazole, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfainethazine, sulfamethizole, sulfamethomidine, sulfamethoxazole, sulfamethoxypyridazine, sulfametrole, sulfamnidochrysoidine, sulfamoxole, sulfanilamide, sulfanilamidomethanesulfonic acid triethanolamine salt, 4-sulfanilamidosalicyclic acid, N₄-sulfanilylsulfanilamide, sulfanilylurea, N-sulfanilyl-3,4-xylamide, sulfanitran, sulfaperine, sulfaphenazole, sulfaproxyline, sulfapyrazine, sulfapyridine, sulfasomizole, sulfasyrnazine, sulfathiazole, sulfathiourea, sulfatolamide, sulfisomidine, sulfisoxazole, acedapsone, acediasulfone, acetosulfone, dapsone, diathymosulfone, glucosulfone, solasulfone, succisulfone, sulfanilic acid, p-sulfanilylbenzylamine, p,p′-sulfonyldianiline-N,N′digalactoside, sulfoxone, thiazolsulfone, salts thereof, derivatives thereof, and mixtures thereof.

In a further alternative preferred embodiment, the antiinfective agent is an aminoglycoside. Preferred, non-limiting examples of aminoglycosides useful in this regard include those selected from the group consisting of amikacin, apramycin, arbekacin, bambeimycins, butirosin, dibekacin, dihydrostreptomycin, fortimicin, fradiomycin, gentamicin, ispamicin, kanamycin, micronomicin, neomycin, netilmicin, paromomycin, ribostamycin, sisomicin, spectinomycin, streptomycin, streptonicozid, tobramycin, salts thereof, derivatives thereof, and mixtures thereof.

In yet another alternative preferred embodiment, the antiinfective agent is a macrolide. Preferred, non-limiting examples of macrolides useful in this regard include those selected from the group consisting of azithromycin, carbomycin, clarithromycin, erythromycin, josamycin, leucomycins, midecamycins, miokamycin, oleandomycin, primycin, rapamycin, rokitamycin, rosaramicin, roxithromycin, spiramycin, troleandomycin, salts thereof, derivatives thereof, and mixtures thereof.

In still yet another alternative preferred embodiment, the antiinfective agent is a penicillin. Preferred, non-limiting examples of penicillins useful in this regard include those selected from the group consisting of amidinocillin, amdinocillin, pivoxil, amoxicillin, ampicillan, apalcillin, aspoxicillin, azidocillan, azlocillan, bacampicillin, benzylpenicillic acid, benzylpenicillin, carbenicillin, carfecillin, carindacillin, clometocillin, cloxacillin, cyclacillin, dicloxacillin, diphenicillin, epicillin, fenbenicillin, floxicillin, hetacillin, lenampicillin, metampicillin, methicillin, mezlocillin, nafcillin, oxacillin, penamecillin, penethamate hydriodide, penicillin G, penicillin N, penicillin O, penicillin V, penimepicycline, phenethicillin, piperacillin, pivapicillin, propicillin, quinacillin, suilbenicillin, talampicillin, temocillin, ticarcillin, salts thereof, derivatives thereof, and mixtures thereof.

In another alternative preferred embodiment, the antiinfective agent is a quinolone. Preferred, non-limiting examples of quinolones useful in this regard include those selected from the group consisting of amifloxacin, cinoxacin, ciprofloxacin, difloxacin, enoxacin, fleroxacin, flumequine, grepafloxacin, levofloxacin, lomefloxacin, miloxacin, nalidixic acid, norfloxacin, ofloxacin, oxolinic acid, perfloxacin, pipemidic acid, piromidic acid, rosoxacin, sparfloxacin, tremafloxacin, trovafloxacin, tosufloxacin, salts thereof, derivatives thereof, and mixtures thereof.

In a further alternative preferred embodiment, the antiinfective agent is an amphenicol. Preferred, non-limiting examples of amphenicols useful in this regard include those selected from the group consisting of azidamfenicol, chloramphenicol, chloramphenicol palmirate, chloramphenicol pantothenate, florfenicol, thiamphenicol, salts thereof, derivatives thereof, and mixtures thereof.

In another further alternative preferred embodiment, the antiinfective agent is a lincosamide. Preferred, non-limiting examples of lincosamides useful in this regard include those selected from the group consisting of clindamycin, lincomycin, salts thereof, derivatives thereof, and mixtures thereof.

In a still further alternative preferred embodiment, the antiinfective agent is an ansamycin. Preferred, non-limiting examples of ansamycins useful in this regard include those selected from the group consisting of rifamide, rifampin, rifamycin, rifaximin, salts thereof, derivatives thereof, and mixtures thereof.

In yet another alternative preferred embodiment, the antiinfective agent is a nitrofuran. Preferred, non-limiting examples of nitrofurans useful in this regard include those selected from the group consisting of furaltadone, furazolium, nifuradene, nifuratel, nifurfoline, nifurpirinol, nifurprazine, nifurtoinol, nitrofurantoin, salts thereof, derivatives thereof, and mixtures thereof.

In still yet another alternative preferred embodiment, the antiinfective agent is a cephamycin. Preferred, non-limiting examples of cephamycins useful in this regard include those selected from the group consisting of cefbuperazone, cefmetazole, cefmninox, cefetan, cefoxitin, salts thereof, derivatives thereof, and mixtures thereof.

In a further alternative preferred embodiment, the antiinfective agent is a monobactam. Preferred, non-limiting examples of monobactams useful in this regard include those selected from the group consisting of aztreonam, carumonam, tigemonan, salts thereof, derivatives thereof, and mixtures thereof.

In still another alternative preferred embodiment, the antiinfective agent is a ketolide. Preferred, non-limiting examples of ketolides useful in this regard include those selected from the group consisting of telithromycin, salts thereof, derivatives thereof, and mixtures thereof.

In another preferred embodiment, the pharmacologically active agent is a steroid. Preferred, non-limiting examples of steroids useful in the present soft gel capsules include those selected from the group consisting of alclometasone dipropionate, amcinonide, beclomethasone dipropionate, betamethasone benzoate, betamethasone dipropionate, betamethasone valerate, budesonide, clobetasol propionate, clobetasone butyrate, cortisone acetate, desonide, desoximetasone, diflorasone diacetate, diflucortolone valerate, fluclorolone acetonide, flumethasone pivalate, fluocinolone acetonide, fluocinonide, fluocortin butyl, fluocortolone preparations, fluprednidene acetate, flurandrenolide, flurandrenolone, fluticosone propionate, halcinonide, halobetasol propionate, hydrocortisone, hydrocortisone acetate, hydrocortisone butyrate, hydrocortisone propionate, hydrocortisone valerate, methylprednisolone acetate, mometasone furoate, pramoxine hydrochloride, prednisone acetate, prednisone valerate, triamcinolone acetonide, salts thereof, derivatives thereof, and mixtures thereof.

In another preferred embodiment, the pharmacologically active agent is an antihistamine. Preferred, non-limiting examples of antihistamine useful in the present soft gel capsules include those selected from the group consisting of acrivastine, AHR 11325, antazoline, astemizole, azatadine, azelastine, bromodiphenhydramine, bromopheniramine, carbinoxamine, cetirizine, chlorpheneramine, clemastine, cyproheptadine, debrompheniramine, descarboethoxyloratadine, desmethylastemizole, dexchlorpheniramine, dimenhydramine, diphenhydramine, diphenylpyraline, doxylamine, ebastine, fexofenadine, hydroxyzine, ketotifen, levocabastine, lodoxamide, loratadine, meclizine, mequitazine, methdilazine, norastemizole, oxatomide, phenindamine, pheniramine promethazine, pseudoephedrine, pyrilamine, setastine, tazifylline, temelastine, terfenadine, trimeprazine, tripelennamine, triprolidine, salts thereof, derivatives thereof, and mixtures thereof.

Controlled Release

In a preferred embodiment, the present soft gel capsules provide a controlled release of the pharmacologically active agent contained therein. This controlled release may be effected by the particular properties of the external layer, as described above. In the alternative, this controlled release may be effected by coating the active ingredient with a release controlling substance. Non-limiting examples of release controlling substances useful in this regard include synthetic or natural oils, waxes, fats, resins, or mixtures thereof.

Preferred pharmaceutically acceptable oils, waxes, fats, or resins useful herein to provide a controlled release of the pharmacologically active agent include but are not limited to microcrystalline waxes, paraffin waxes, carnauba waxes, fatty acids and their salts, mono and diglyceride salts, esters of mono and diglycerides, agars, agaroses, algins, low methoxy pectins, gellans, K-carrageenan, t-carrageenan, furcellaran, β-carrageenan, curdlan, chitosan, konjac glucomannan and derivatives thereof including heat stable cold-melt knjac glucomannan, cellulose derivatives, starches, and mixtures of two or more of the foregoing, as well as hydrocolloid mixtures such as xanthan/locust bean gum, locust bean gumlagar, cassia/agar, cassia/xanthan, konjac/xanthan, carrageenan/locust bean gum, konjac/carrageenan, konjac/starch, other suitable waxes, fats, or resins, and mixtures thereof.

Another non-limiting way to produce the controlled release pharmacologically active agent used in the present compositions is to disperse this material in a matrix by first liquefying the matrix material with heat and then dispersing the active agent with a shearing or mixing operation. The active agent remains dispersed in the matrix material as the matrix material solidifies or congeals.

Methods of Treatment

The softgel, or soft gelatin, capsules provided herein are preferably administered to a mammal in order to treat a dermatological disease or disorder in the mammal. In a preferred embodiment, the method of treatment is accomplished by orally administering the soft gel capsule to a mammal to effectively treat a dermatological disease or disorder. Preferred dermatological disorders treatable according to the present methods include primary and secondary skin infections.

In preferred embodiments, the mammal being treated is a human. In particularly preferred embodiments in this regard, the human being treated is a human child of between 5 and 20 years old, preferably a human child of between 8 and 18 years old, a human of at least 55 years of age, or a human female.

Several specific dermatological diseases or disorders may be treated according to the present methods. Exemplary among these dermatological diseases or disorders are those selected from the group consisting of bacterial infections of the skin, dermatitis, disorders of hair follicles and sebaceous glands, fungal skin infections, parasitic skin infections, pruritis, hyperpigmentary diseases, hypopigmentary diseases, hyperproliferative cell disorders, scaling papular diseases, and combinations thereof. Other dermatological diseases or disorders known as effectively treatable by any of the pharmacologically active agents described herein are further contemplated as treatable according to the present methods.

In a particularly preferred embodiment, the dermatological disorder to be treated according to the present methods is dermatitis. Preferred, non-limiting forms of dermatitis treatable according to the present methods include those selected from the group consisting of contact dermatitis, allergic contact dermatitis, atopic dermatitis, seborrheic dermatitis, nummular dermatitis, chronic dermatitis of the hands and feet, generalized exfoliative dermatitis, stasis dermatitis, lichen complex dermatitis, and combinations thereof.

In an alternative particularly preferred embodiment, the dermatological disorder to be treated according to the present methods is a disorder of hair follicles and sebaceous glands. Preferred, non-limiting examples of disorders of hair follicles and sebaceous glands treatable according to the present methods include those selected from the group consisting of acne, rosacea, perioral dermatitis, hypertrichosis, alopecia, pseudofolliculitis barbae, keratinous cysts, and combinations thereof.

In another alternative particularly preferred embodiment, the dermatological disorder to be treated according to the present methods is a scaling popular disease. Preferred, non-limiting examples of scaling popular diseases treatable according to the present methods include those selected from the group consisting of psoriasis, pityriasis rosea, lichen planus, pityriasis rubra pilaris, and combinations thereof.

Particularly preferred dermatological disorders treatable according to the present methods are dermatitis, pruritis, and acne.

The present soft gel capsules may additionally be used in treating alternative diseases, disorders, or conditions, such as for various cough and cold uses.

Combination Therapy

In another preferred embodiment, the present soft gel capsules may be used in combination with an additional pharmaceutical dosage form to enhance the effectiveness in treating a dermatological disease or disorder. In this regard, the present soft gel capsules may be administered as part of a regimen additionally including any other pharmaceutical and/or pharmaceutical dosage form known in the art as effective for the treatment of a dermatological disorder. Similarly, an active ingredient other than those specified herein can be added to the present soft gels to enhance their effectiveness in treating a dermatological disease or disorder. Accordingly, this additional active ingredient or additional pharmaceutical dosage form can be applied to a patient either directly or indirectly, and concomitantly or sequentially, with the soft gel capsules described herein.

In one embodiment in this regard, the present soft gel capsules and the additional pharmaceutical dosage form can be administered to a patient at the same time. In an alternative embodiment, one of the present soft gel capsules and the additional pharmaceutical dosage form can be administered in the morning and the other can be administered in the evening.

Methods of Production

The present soft gel capsules can be produced by methods well known in the art for the preparation of soft gel oral dosage forms, i.e. by encapsulating the fill material between two sheets of gelatin as it passes between a pair of die rolls having surface cavities shaped to form the desired shape of the resulting softgel.

The first step according to one process for preparing the present soft gel capsules is the production of the internal liquid phase. This internal liquid phase may be prepared with proper specification mixing, refining, and homogenizing equipment and procedures, sometimes under vacuum. The particle size of the pharmacologically active agent included in the internal liquid phase, as well as the viscosity of the internal liquid phase, is optimized for easy and accurate encapsulation. The internal liquid phase should be produced so as to avoid cross-contamination between products.

Once the internal liquid phase has been prepared, the next process step is to prepare the capsule base, or external layer. This step is important as it directly affects the soft gel capsule shape, appearance, and seam strength. The external layer can be prepared according to any process commonly known in the art.

The most critical step of this process is the encapsulation of the internal liquid phase in the external layer. One preferred encapsulation process is a rotary die process, which is a continuous single operation. This process is preferred as it forms and fills the softgel capsule in a single step under conditions of low humidity and accurately controlled temperature.

According to one contemplated rotary die process, the encapsulation process begins when molten gel is pumped to the machine and thin ribbons of gel are formed on either side of the machine. These ribbons then pass over a series of rollers and over a set of die that determine the size and shape of the capsules. The internal liquid phase is then fed to a positive displacement pump, which accurately doses the internal liquid phase and injects it between two gelatin ribbons prior to sealing them together through the application of heat and pressure. The capsules formed at this stage are incredibly flexible due to water in the gel mass.

Following encapsulation, softgels typically undergo drying. Such drying may be conducted by any combination of tumble drying, fluid bed drying, and tray drying, as well as any other drying procedures known in the art. For example, the capsules may pass into tumble dryers where about 25% of water is removed. The capsules are then placed on trays, which are stacked and transferred to drying rooms where dry air is forced over the capsules to remove any excess moisture. The moisture is measured at regular intervals. When the moisture is limited to approximately 8%, the drying process is complete and the capsules are ready for packaging. P. Tyle, Specialized Drug Delivery Systems, Marcel Dekker, Inc. (1990); M. S. Patel et al., “Advances in Softgel Formulation Technology”, Manufacturing Chemists, July 1989; William R. Ebert, “Soft Elastic Gelatin Capsules: A Unique Dosage Form”, Pharmaceutical Technology, October 1977; H. Seager, “Soft gelatin capsules: a solution to many tableting problems”, Pharmaceutical Technology, September 1985; and U.S. Pat. Nos. 4,067,960, 4,198,391, 4,744,988, 4,780,316, and 5,200,191, the entire contents of which are hereby incorporated by reference, provide a general discussion of this and other processes used in softgel manufacturing.

Further contemplated as within the scope of the present subject matter are pharmaceutical compositions produced according to the above-described process. If produced according to this process, these compositions exhibit chemical and physical stability suitable for oral administration.

Dosage

Appropriate dosage levels for the pharmacologically active agents contemplated herein are well known to those of ordinary skill in the art. Dosage levels on the order of about 0.001 mg to about 5,000 mg per kilogram body weight of the pharmacologically or dermatologically active agent are known to be useful in the treatment of the diseases, disorders, and conditions contemplated herein. Typically, this effective amount of the pharmacologically active agent will generally comprise from about 0.1 mg to about 100 mg per kilogram of patient body weight per day. Moreover, it will be understood that this dosage of active therapeutic agents can be administered in a single or multiple dosage units to provide the desired therapeutic effect.

If desired, other therapeutic agents can be employed in conjunction with those provided above. The amount of active ingredients that may be combined with the carrier materials to produce a single dosage form will vary depending upon the host treated, the nature of the disease, disorder, or condition, and the nature of the active ingredients.

The present compositions may be given in a single or multiple doses daily. In a preferred embodiment, the present compositions are given from one to three times daily. Starting with a low dose twice daily and slowly working up to higher doses if needed is a preferred strategy. The amount of active ingredients that may be combined with the carrier materials to produce a single dosage form will vary depending upon the host treated, the nature of the disease, disorder, or condition, and the nature of the active ingredients.

It is understood, however, that a specific dose level for any particular patient will depend upon a variety of factors well known in the art, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

The optimal pharmaceutical formulations will be determined by one skilled in the art depending upon considerations such as the particular drug or drug combination and the desired dosage. See, for example, “Remington's Pharmaceutical Sciences”, 18 th ed. (1990, Mack Publishing Co., Easton, Pa. 18042) and “Harry's Cosmeticology”, 8 th ed. (2000, Chemical Publishing Co., Inc., New York, N.Y. 10016), the entire disclosures of which are hereby incorporated by reference. Such formulations may influence the physical state, stability, rate of in vivo release, and rate of in vivo clearance of the therapeutic agents.

### EXAMPLES

The following examples are illustrative of the present subject matter and are not intended to be limitations thereon. All polymer molecular weights are mean average molecular weights. All percentages are based on the percent by weight of the final delivery system or formulation prepared unless otherwise indicated and all totals equal 100% by weight.

### Example 1

The following example illustrates the preparation of a preferred soft gel capsule of the present subject matter:

| | |
|---|---|
| External Phase | |
| Gelatin | 168.0 mg |
| Glycerin | 52.0 mg |
| Sorbitol/Sorbitane | 43.0 mg |
| Water | 137.0 mg |
| Internal Phase | |
| Polyethylene Glycol 600 | 459.0 mg |
| Glycerin | 51.0 mg |
| Water | 38.0 mg |
| Doxycycline monohydrate | 100.0 mg |

1. An internal liquid phase is prepared by mixing the polyethylene glycol, glycerin, water, and doxycycline monohydrate until homogenous.

2. In a separate container, a molten gel is produced by mixing the gelatin, glycerin, sorbital/sorbitane, and water until a homogenous molten gel has formed.

3. The molten gel is then pumped through a rotary die machine to form thin ribbons of gel on either side of this machine. These ribbons are passed over a series of rollers and a set of die to prepare an oval capsule shape. The internal liquid phase is then fed to a positive displacement pump, which injects the internal liquid phase between two gelatin ribbons. The gelatin ribbons are then sealed together via heat and pressure. These capsules are then dried to remove moisture from the external phase.

### Example 2

The following example illustrates the preparation of another soft gel capsule of the present subject matter:

| | % W/W |
|---|---|
| External Phase | |
| Gelatin | 38.5 |
| Glycerol | 20.7 |
| Anidrisorb ™ 35/70 | 8.8 |
| Water | 32.0 |
| | 100.0% |

* Andrisorb ™ is a proprietary mix of sorbitol, sorbitan and
mannitol available from Roquette Freres

| | |
|---|---|
| Internal Phase | |
| Doxycycline hyclate | 8.00 |
| Labrosol ® | 31.30 |
| Plurol ® Oleique CC497 | 13.26 |
| Labrafac ® CC | 39.00 |
| Water | 8.44 |
| | 100.0% |

* Labrosol ® is a proprietary caprylocaproyl macrogol-8 glyceride,
Plurol ® Oleique CC497 is a proprietary polyglycerol-6 diolate, and
Labrafac ® CC is a proprietary medium chain triglyceride, all available
from Gattefossé Pharmaceutical division.

1. An internal liquid phase is prepared by mixing the Labrosol®, Plurol® Oleique CC497, Labrfac® CC, water and doxycycline hyclate until homogenous.

2. In a separate container, a molten gel is produced by mixing the gelatin, glycerol, Andrisorb 35/70, and water until a homogenous molten gel has formed.

3. The molten gel is then pumped through a rotary die machine to form thin ribbons of gel on either side of this machine. These ribbons are passed over a series of rollers and a set of die to prepare an oval capsule shape. The internal liquid phase is then fed to a positive displacement pump, which injects the internal liquid phase between two gelatin ribbons. The gelatin ribbons are then sealed together via heat and pressure. These capsules are then dried to remove moisture from the external phase.

### Example 3

A patient is suffering from dermatitis. A soft gel capsule as herein described is orally administered to the patient. It would be expected that the patient would improve his/her condition or recover.

### Example 4

A patient is suffering from acne. A soft gel capsule as herein described is orally administered to the patient. It would be expected that the patient would improve his/her condition or recover.

### Example 5

A patient is suffering from pruritis. A soft gel capsule as herein described is orally administered to the patient. It would be expected that the patient would improve his/her condition or recover.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

1. A method of treating a dermatological disorder in a mammal, comprising:
orally administering to said mammal a soft gel capsule providing improved bioavailability of a pharmacologically active agent comprising:
an internal, non-aqueous liquid phase comprising a solution or suspension of a single, hydrophobic, pharmacologically active agent effective to treat said dermatological disorder having a purity of at least 90% and a concentration of degradation product(s) less than about 10% of the starting concentration of said hydrophobic pharmacologically active agent, wherein said purity and concentration of degradation product(s) are sufficient to permit safe treatment of said dermatological disorder; and
an external gelatin layer comprising gelatin, soft cellulose, or a mixture thereof and additional components selected from the group consisting of an additional gelling agent, a plasticizer, water, a colorant, an antioxidant, a flavorant, and mixtures thereof;
wherein said hydrophobic pharmacologically active agent is selected from the group consisting of antiinfectives, steroids, a salt thereof, a derivative thereof, and mixtures thereof.

2. The method of claim 1, wherein said pharmacologically active agent has a purity of at least 95%.

3. The method of claim 1, wherein said internal liquid phase maintains a concentration of degradation product(s) less than about 7% of the starting concentration of said pharmacologically active agent.

4. The method of claim 3, wherein said internal liquid phase maintains a concentration of degradation product(s) less than about 5% of the starting concentration of said pharmacologically active agent.

5. The method of claim 1, wherein said external layer permits easier swallowing of said soft gel capsule in comparison to hard gelatin capsules.

6. The method of claim 1, wherein said mammal is a human.

7. The method of claim 6, wherein said human is a human child of between 5 and 20 years old or a human of between 55 and 90 years old or older.

8. The method of claim 7, wherein said human child has an age of between 8 and 18 years old.

9. The method of claim 6, wherein said human is a female.

10. The method of claim 1, wherein said external layer provides a controlled release of the pharmacologically active agent.

11. The method of claim 1, wherein said soft gel capsule improves palatability of the pharmacologically active agent.

12. The method of claim 11, wherein said improved palatability results in improved patient compliance with said administration of said pharmacologically active agent.

13. The method of claim 1, wherein said soft gel capsules provide a reduced incidence of side effects of the pharmacologically active agent upon administration to said mammal.

14. The method of claim 1, wherein said internal liquid phase has a pH of about 3 to about 9 when combined with an aqueous medium.

15. The method of claim 1, wherein said internal liquid phase further comprises one or more fatty acids or derivatives thereof selected from the group consisting of fatty acids, esters of fatty acids, ethers of fatty acids, alcohols of fatty acids, and mixtures thereof.

16. The method of claim 1, wherein said internal liquid phase further comprises an additional ingredient selected from the group consisting of peanut oil, hydrogenated peanut oil, castor oil, hydrogenated castor oil, corn oil, olive oil, hydrogenated vegetable oils, silicone oil, soya oil, paraffin oil, cetyl alcohol, cetostearyl alcohol, stearyl alcohol, stearic acid, beeswax, silica dioxide, polyethylene glycol, monoglycerides, diglycerides, triglycerides, poloxamers, and mixtures thereof.

17. The method of claim 1, wherein said dermatological disorder is selected from the group consisting of primary and secondary skin infections.

18. The method of claim 1, wherein said dermatological disorder is selected from the group consisting of bacterial infections of the skin, dermatitis, disorders of hair follicles and sebaceous glands, fungal skin infections, parasitic skin infections, pruritis, hyperpigmentary diseases, hypopigmentary diseases, hyperproliferative cell disorders, scaling papular diseases, and combinations thereof.

19. The method of claim 1, wherein said antiinfective is a tetracycline or a salt or derivative thereof.

20. The method of claim 19, wherein said tetracycline is doxycycline or a salt or derivative thereof.

21. The method of claim 1, wherein said soft gel capsules are administered concomitantly or sequentially with an additional pharmaceutical dosage form effective to treat said dermatological disorder.

22. A method of treating a dermatological disorder in a mammal, comprising:
orally administering to said mammal a soft gel capsule providing improved bioavailability of a pharmacologically active agent comprising:
an internal, non-aqueous liquid phase having a pH of from about 3 to about 9 when combined with an aqueous medium comprising a solution or suspension of a single, hydrophobic, pharmacologically active agent effective to treat said dermatological disorder and one or more fatty acids or derivatives thereof selected from the group consisting of omega-3 fatty acids, DHA, docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, said single pharmacologically active agent comprising a hydrophobic antiinfective agent or a salt or derivative thereof having a purity of at least 90% and a concentration of degradation product(s) less than about 10% of the starting concentration of said hydrophobic antibiotic agent, wherein said purity and concentration of degradation product(s) are sufficient to permit safe treatment of said dermatological disorder; and
an external gelatin layer comprising gelatin and additional components selected from the group consisting of an additional gelling agent, a plasticizer, water, a colorant, an antioxidant, a flavorant, and mixtures thereof.

23. The method of claim 22, wherein said hydrophobic antiinfective agent has a purity of at least 95%.

24. The method of claim 22, wherein said internal liquid phase maintains a concentration of degradation product(s) less than about 7% of the starting concentration of said hydrophobic antiinfective agent.

25. A method of treating a dermatological disorder in a mammal, comprising:
orally administering to said mammal a soft gel capsule providing improved bioavailability of doxycycline or a salt or derivative thereof comprising:
an internal, non-aqueous liquid phase having a pH of from about 3 to about 9 when combined with an aqueous medium comprising a solution or suspension of doxycycline or a salt or derivative thereof as a sole active ingredient effective to treat said dermatological disorder and one or more fatty acids or derivatives thereof selected from the group consisting of omega-3 fatty acids, DHA, docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, said doxycycline having a purity of at least 95% and a concentration of degradation product(s) less than about 5% of the starting concentration of said doxycycline, wherein said purity and concentration of degradation product(s) are sufficient to permit safe treatment of said dermatological disorder; and
an external gelatin layer comprising gelatin and additional components selected from the group consisting of an additional gelling agent, a plasticizer, water, a colorant, an antioxidant, a flavorant, and mixtures thereof.
